(19) United States
(12) Patent Application Publication
(10) Pub. No.: US 2007/0255219 A1
(43) Pub. Date: Nov. 1, 2007

(54) Instrument access device

(76) Inventors: Trevor Vaugh, Birr (IE); John Butler, Blackrock (IE); Shane J. MacNally, Delgany (IE); Frank Bonadio, Bray (IE)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/785,752

(22) Filed: Apr. 19, 2007

Related U.S. Application Data
(60) Provisional application No. 60/792,954, filed on Apr. 19, 2006.

(51) Int. Cl. A61M 5/178

(52) U.S. Cl. 604/167.02

(57) ABSTRACT

An instrument access device (1) comprises an instrument seal, a wound retractor for retracting a wound opening (2), and an opening element for at least partially opening the seal to facilitate insertion of an instrument (6) through the seal. The instrument seal comprises a gelatinous elastomeric seal (3) with a pinhole opening (4) extending therethrough, and a housing (5) around the seal (3). The opening element may be employed to at least partially open the pinhole opening (4) to facilitate insertion of the instrument (6) through the pinhole opening (4). The opening element comprises a sheet (11) of flexible material. The sheet (11) is movable distally relative to the seal (3) between a rest configuration and an opening configuration. In the rest configuration the sheet (11) is substantially planar. In the opening configuration, the pointed, the leading end (16) of the sheet (11) enters the pinhole opening (4) and moves distally through the pinhole opening (4) to at least partially open the pinhole opening (4).

### INTRODUCTION

This invention relates to an instrument access device, and to a method of accessing a wound interior with an instrument.

### STATEMENTS OF INVENTION

According to the invention there is provided an instrument access device comprising:—
at least one instrument seal; and
at least one opening element to at least partially open the at least one instrument seal for insertion of an instrument through the at least one instrument seal.

By using the opening element to open the seal, this arrangement minimises the possibility of the instrument damaging the seal. In particular the instrument itself may not be required to engage against the seal to open the seal.

In one embodiment of the invention at least part of the opening element is movable relative to the seal to open the seal. The opening element may be movable distally relative to the seal. The opening element may be movable between a rest configuration and an opening configuration. In the opening configuration, at least part of the opening element may be substantially pointed. In the opening configuration, at least part of the opening element may be substantially tapered. In the opening configuration, at least part of the opening element may be substantially conical-shaped. In the rest configuration, the opening element may be substantially planar.

In one case the opening element is attached to the seal. The device may comprise a connector element to attach the opening element to the seal.

In another case the opening element is detached from the seal.

In one embodiment the opening element comprises a sheet of material. In the opening configuration, a first part of the sheet may overlap a second part of the sheet. By overlapping the first part over the second part, this enables the sheet to move between a planar configuration and a pointed/tapered/conical configuration. The sheet may comprise a discontinuity between the first part and the second part. The discontinuity may comprise a slit in the sheet.

In one case the opening element is substantially flexible. In another case the opening element is substantially rigid.

The device may comprise a plurality of opening elements.

In one embodiment the seal comprises a gelatinous elastomeric material. The seal may comprise at least one opening extending therethrough through which an instrument may be inserted. The opening element may be configured to at least partially open the opening for insertion of an instrument through the opening. The opening may be biased towards a closed configuration. The opening may comprise a pinhole opening.

The invention also provides in another aspect an instrument access device comprising:—
at least one instrument seal;
the at least one instrument seal comprising at least one opening extending at least partially therethrough through which an instrument may be inserted; and
at least one reinforcement element to reinforce the at least one instrument seal upon engagement of an instrument with the at least one instrument seal;
the at least one reinforcement element being located radially outwardly of the opening.

By reinforcing the seal, this arrangement minimises the possibility of the instrument damaging the seal.

In one embodiment of the invention, the reinforcement element is located distally of a proximal end of the seal. The reinforcement element may be located proximally of a distal end of the seal. The reinforcement element may be at least partially embedded within the seal.

In one case the reinforcement element is of a more rigid material than the seal. The seal may comprise a gelatinous elastomeric material.

The opening may comprise a lipseal opening. The device may comprise a plurality of reinforcement elements. The reinforcement elements may be circumferentially spaced-apart around the opening.

In another embodiment the device comprises:—
a distal anchoring member for insertion into a wound interior; and
an elongate member extending proximally from the distal anchoring member to retract laterally the sides of a wound opening.

The distal anchoring member acts to maintain the device in position relative to the wound opening. This may be particularly useful in the case where the wound interior is insufflated, in which case there may be a high pressure within the wound interior.

In one case the device comprises a proximal member for location externally of a wound opening. The proximal member may comprise a ring member. The proximal member may comprise a proximal inner element and a proximal outer element. The elongate member may be led between the proximal inner element and the proximal outer element. The proximal inner element and/or the proximal outer element may comprise a ring element. The proximal outer element may be mounted to the proximal inner element.

In another aspect of the invention there is provided a method of accessing a wound interior with an instrument, the method comprising the steps of:—
creating a wound opening;
inserting a distal anchoring member into the wound interior;
retracting the sides of the wound opening;
arranging an instrument seal at the wound opening;
at least partially opening the instrument seal; and
inserting an instrument through the instrument seal to access the wound interior.

In one embodiment the seal is opened using an opening element. The method may comprise the step of moving at least part of the opening element relative to the seal to open the seal. At least part of the opening element may be moved distally relative to the seal. The method may comprise the step of engaging the instrument with the opening element to move at least part of the opening element relative to the seal.

In one case the sides of the wound opening are retracted using an elongate member extending proximally from the distal anchoring member.

### BRIEF DESCRIPTION OF THE DRAWINGS

The invention will be more clearly understood from the following description of some embodiments thereof, given by way of example only, with reference to the accompanying drawings, in which:—

FIG. 1 is a cross-sectional, side view of an instrument access device according to the invention;

FIG. 2 is a plan view of the device of FIG. 1;

FIG. 3 is an isometric view of an opening element part of the device of FIG. 1;

FIGS. 4 and 5 are views similar to FIG. 1 of the device of FIG. 1, in use;

FIG. 6 is an isometric view of the opening element part of FIG. 3, in use;

FIGS. 7 to 9 are views similar to FIG. 1 of the device of FIG. 1, in use;

FIGS. 10 to 12 are views similar to FIG. 1 of another instrument access device according to the invention, in use;

FIG. 13 is a plan view of another instrument access device according to the invention;

FIGS. 14 to 16 are views similar to FIG. 1 of the device of FIG. 13, in use;

FIGS. 17 to 20 are views similar to FIG. 1 of a further instrument access device according to the invention, in use;

FIGS. 21 to 25 are views similar to FIG. 1 of another instrument access device according to the invention, in use;

FIGS. 26 to 28 are views similar to FIG. 1 of a further instrument access device according to the invention, in use;

FIG. 29 is an isometric view of another instrument access device according to the invention;

FIG. 30 is a plan view of the device of FIG. 29; and

FIG. 31 is a cross-sectional, side view of the device of FIG. 29.

### DETAILED DESCRIPTION

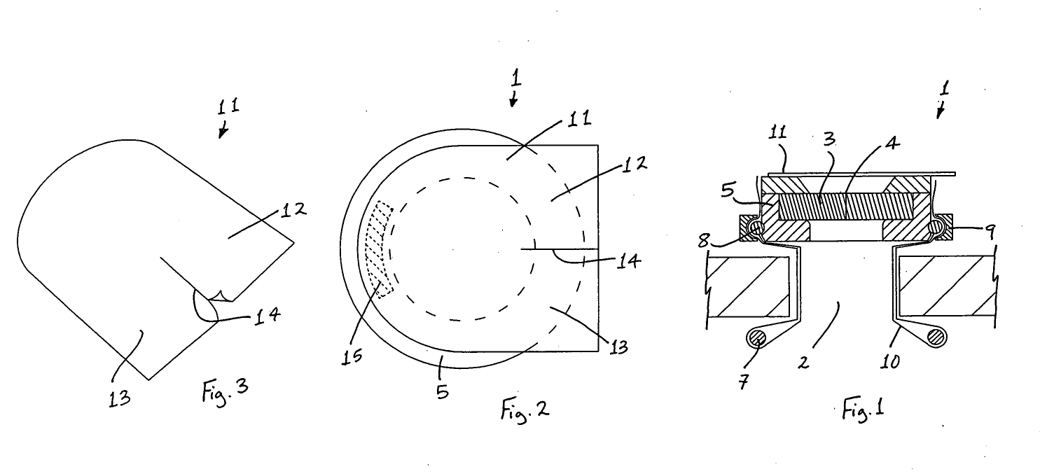

Referring to the drawings, and initially to FIGS. 1 to 9 thereof, there is illustrated an instrument access device 1 according to the invention. The device 1 comprises an instrument seal, a wound retractor for retracting a wound opening 2, and an opening element for at least partially opening the seal to facilitate insertion of an instrument 6 through the seal.

The instrument seal comprises a gelatinous elastomeric seal 3 with a pinhole opening 4 extending therethrough, and a housing 5 around the seal 3. The pinhole opening 4 is biased towards a closed configuration (FIG. 1). The opening element may be employed to at least partially open the pinhole opening 4 to facilitate insertion of the instrument 6 through the pinhole opening 4 (FIG. 7).

The wound retractor comprises a distal anchoring O-ring 7 for insertion into a wound interior, a proximal inner O-ring 8 for location externally of the wound opening 2, a proximal outer ring element 9 for location externally of the wound opening 2, and a elongate sleeve 10 for retracting laterally the sides of the wound opening 2, the sleeve 10 extending between the distal O-ring 7 and the proximal rings 8, 9 in a double layer. In particular a first end of the sleeve 10 is attached to the proximal inner O-ring 8 and the sleeve 10 extends distally as an inner layer from the proximal inner O-ring 8 to the distal O-ring 7. The sleeve 10 is looped around the distal O-ring 7 and extends proximally as an outer layer to the proximal rings 8, 9. The sleeve 10 extends proximally between the proximal rings 8, 9 to a second end of the sleeve 10. The proximal outer ring element 9 is mounted to the proximal inner O-ring 8.

The opening element comprises a sheet 11 of flexible material. The sheet 11 has a first corner part 12 and a second corner part 13, with a slit 14 between the first part 12 and the second part 13. The sheet 11 is attached to the seal housing 5 at an attachment region 15 (FIG. 2). The attachment region 15 is located at the opposite side of the sheet 11 to the slit 14. As illustrated in FIG. 2, the attachment region 15 extends around only part of the circumference of the housing 5, in this case around approximately 45°.

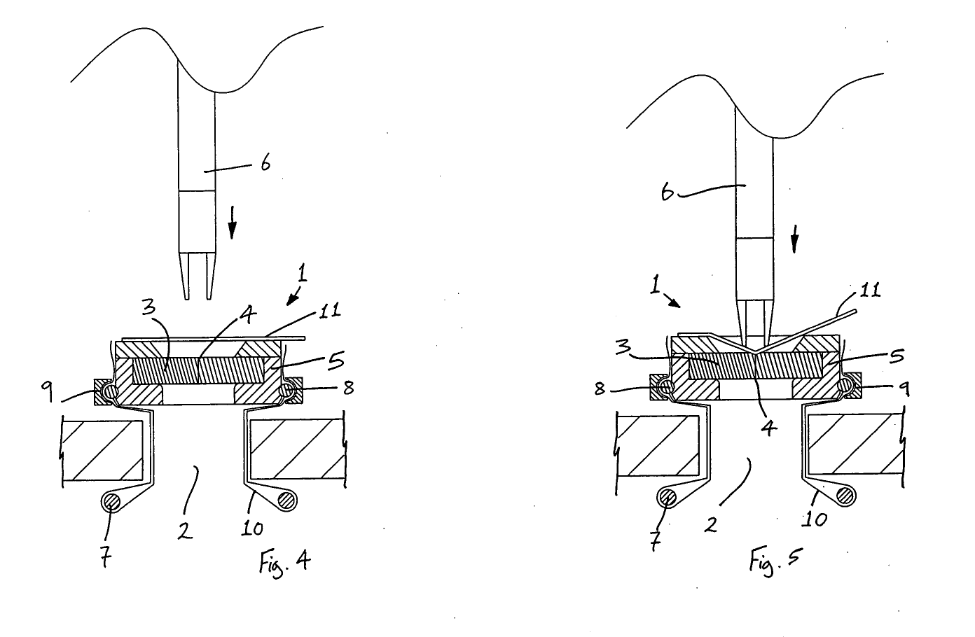

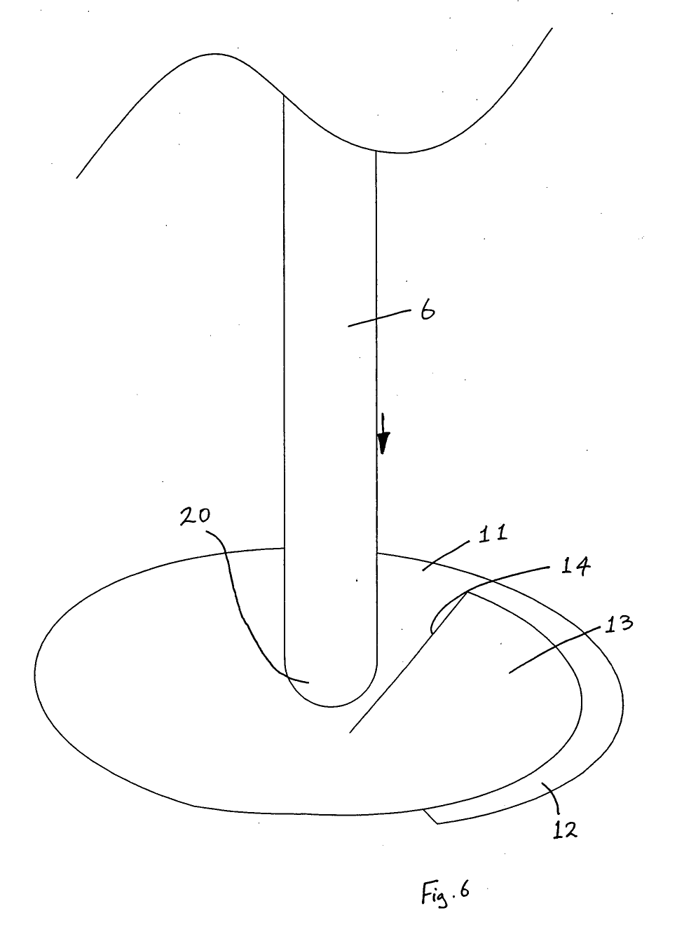

The sheet 11 is movable distally relative to the seal 3 between a rest configuration (FIGS. 1 and 4) and an opening configuration (FIGS. 6 and 7). In the rest configuration the sheet 11 is substantially planar and the sheet 11 is not in contact with the seal 3. In the opening configuration the first corner part 12 overlaps the second corner part 13 to form the sheet 11 into a substantially pointed, tapered, conical shape. In the opening configuration, the pointed, leading end 16 of the sheet 11 may enter the pinhole opening 4 and move distally through the pinhole opening 4 to at least partially open the pinhole opening 4.

In use, the wound opening 2 is created and the distal anchoring O-ring 7 is inserted through the wound opening 2 into the wound interior. The proximal rings 8, 9 and the instrument seal are arranged externally of the wound opening 2. The free second end of the sleeve 10 is pulled proximally to retract the sides of the wound opening 2 (FIG. 1).

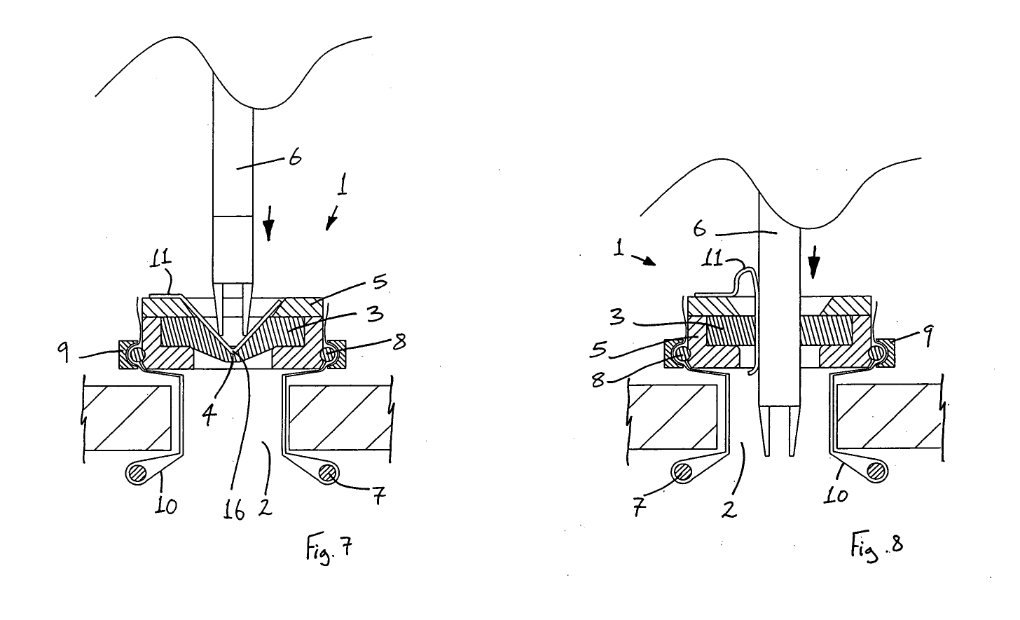

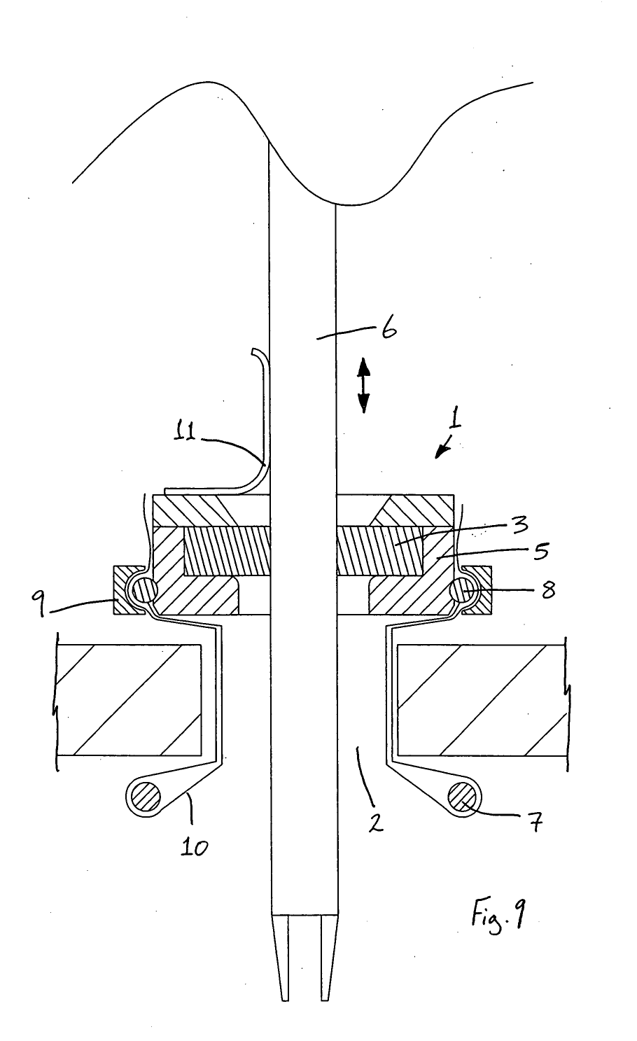

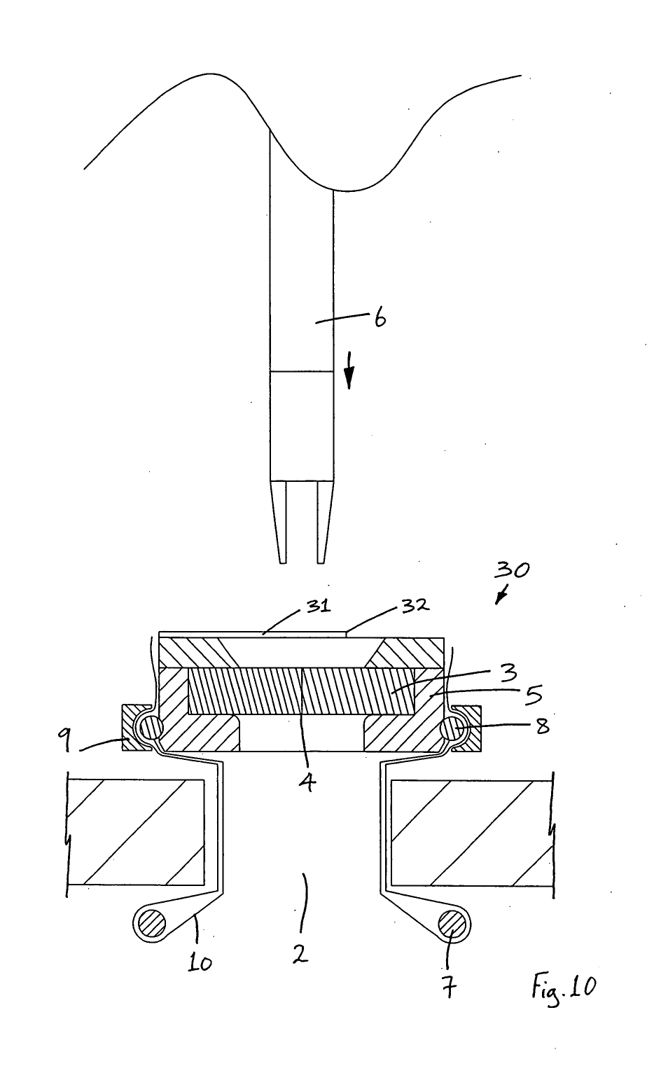

Upon advancement of the instrument 6 distally (FIG. 4), the instrument 6 engages the sheet 11, and moves the sheet 11 distally relative to the seal 3 from the rest configuration to the opening configuration (FIGS. 5 and 7). In particular in the opening configuration, the pointed, leading end 16 of the sheet 11 enters the pinhole opening 4 and moves distally through the pinhole opening 4 to at least partially open the pinhole opening 4. Because the pinhole opening 4 is at least partially opened, it is easier for the instrument 6 to pass through the pinhole opening 4 without the risk of the instrument 6 inadvertently piercing or ripping the seal 3 to the side of the pinhole opening 4. The instrument 6 is then advanced distally through the at least partially opened pinhole opening 4, through the retracted wound opening 2 to access the wound interior (FIG. 8). Reciprocating motion of the instrument 6 distally/proximally causes the sheet 11 to evert proximally out of the pinhole opening 4 (FIG. 9).

It is possible to increase the lubrication of the device 1 by lubricating the proximal surface of the sheet 11 in addition to the seal 3 at the pinhole opening 4.

The conical shape of the sheet 11 in the opening configuration assists in guiding the instrument 6 to find the pinhole opening 4 to prevent ripping/piercing of the seal 3.

The device 1 is suitable for use with a variety of instruments 6, for example instruments having smooth tips or instruments having sharpened/pointed tips.

The buttonhole flap 11 may be a thin fabric, for example a 50/70 micron nylon with PU coating (FIG. 3). The sheet 11 is fixed to the top of the gel housing 5, for example by welding, or gluing (FIG. 2). The sheet 11 protects the gel 3 from the prongs of the clip applier 6 (FIG. 5). FIG. 6 illustrates any shaped instrument tip 20. In 3D, the buttonhole flap 11 forms a cone. The slit 14 facilitates the formation of this cone. The buttonhole flap 11 finds the pinhole 4, and spreads the pinhole 4, and the clip applier 6 follows through (FIG. 7). A slight jiggling of the instrument 6, which may occur naturally during normal use, causes the buttonhole flap 11 to evert back out of the gel valve 3 (FIG. 9).

In FIGS. 10 to 12 there is illustrated another instrument access device 30 according to the invention, which is similar to the device 1 of FIGS. 1 to 9, and similar elements in FIGS. 10 to 12 are assigned the same reference numerals.

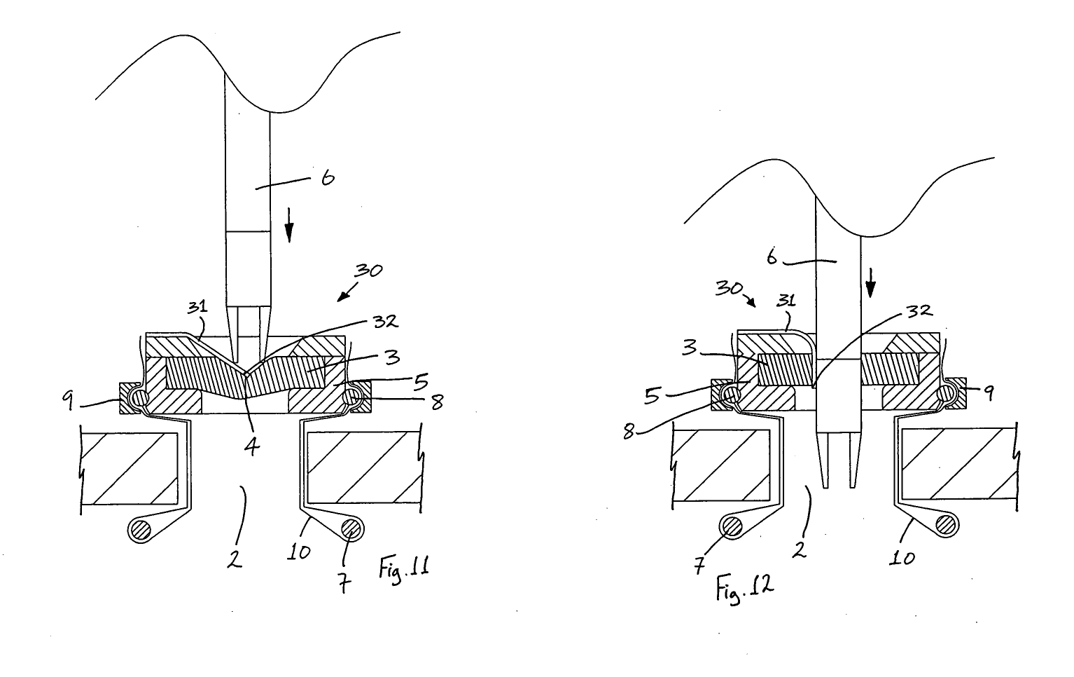

In this case the opening element comprises a substantially rigid finger 31. The finger 31 extends across only part of the width of the seal 3 in the rest configuration (FIG. 10). In the opening configuration, the free end 32 of the finger 31 enters the pinhole opening 4 and moves distally through the pinhole opening 4 to at least partially open the pinhole opening 4 (FIG. 11).

FIG. 10 illustrates the gel protector 31 which is fixed to one side at the top of the gel housing 5. The gel protector 31 flexes upon contact from the instrument 6 and deflects, and begins to open the pinhole 4 in the gel 3 (FIG. 11). The instrument 6 now easily finds the splayed gel pinhole 4 and drives through (FIG. 12).

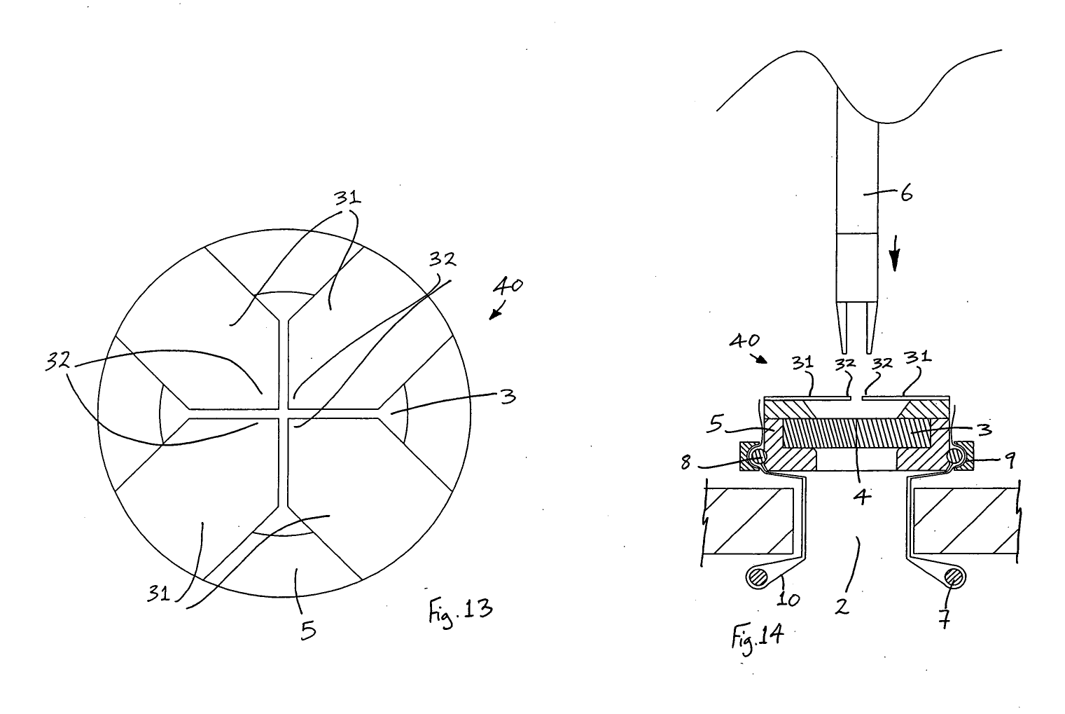

FIGS. 13 to 16 illustrate another instrument access device 40 according to the invention, which is similar to the device 30 of FIGS. 10 to 12, and similar elements in FIGS. 13 to 16 are assigned the same reference numerals.

In this case the device 40 comprises four rigid fingers 31 arranged circumferentially spaced-apart around the seal 3.

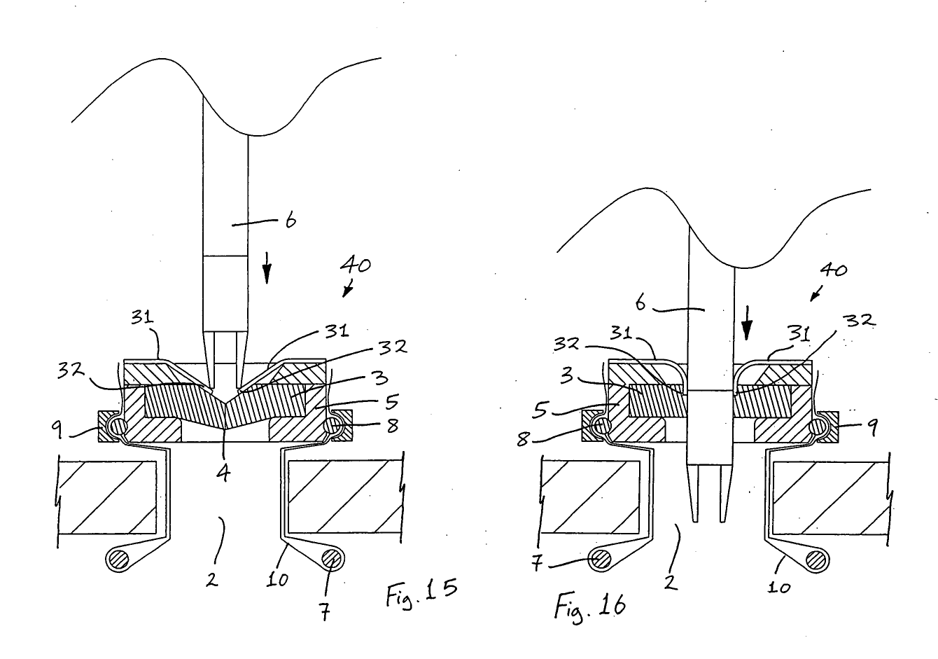

FIG. 13 illustrates the four concentrically mounted gel protectors 31. The gel protectors 31 deflect downwards under pressure from the advancing instrument 6, and thus the pinhole 4 becomes splayed (FIG. 15). The instrument 6 can now easily find the pinhole 4 and drive through (FIG. 16).

Referring to FIGS. 17 to 20 there is illustrated another instrument access device 50 according to the invention, which is similar to the device 1 of FIGS. 1 to 9, and similar elements in FIGS. 17 to 20 are assigned the same reference numerals.

In this case the sheet 11 is detached from the seal housing 5.

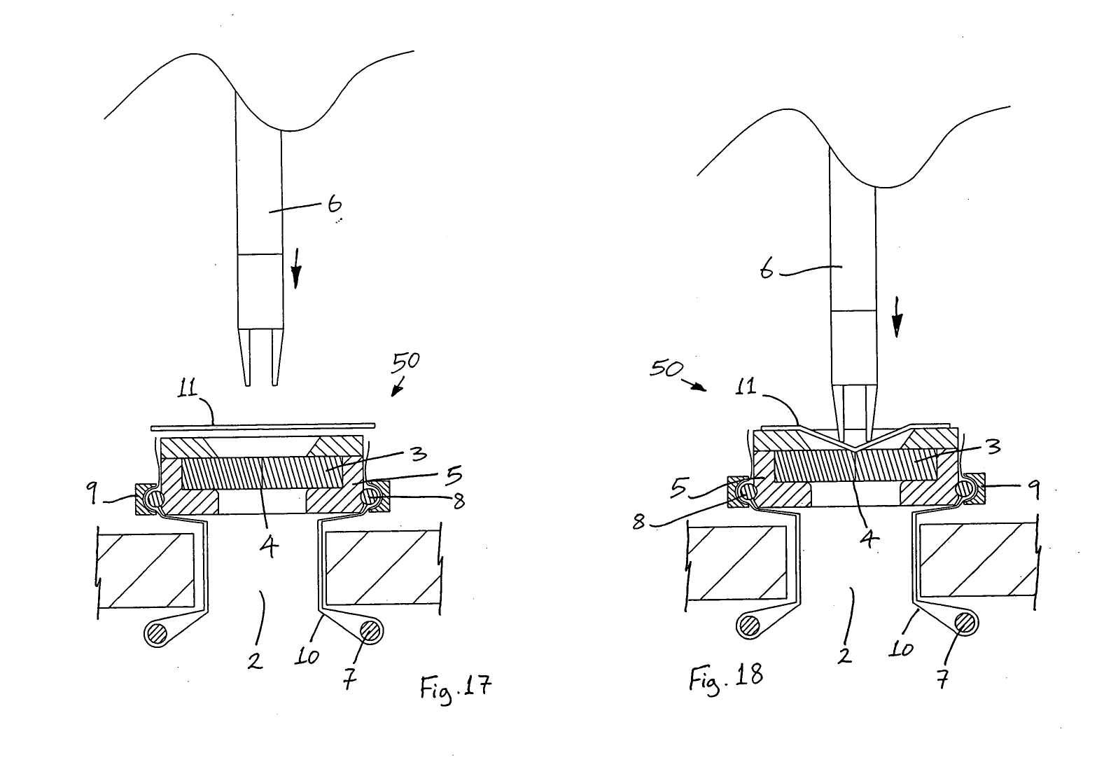

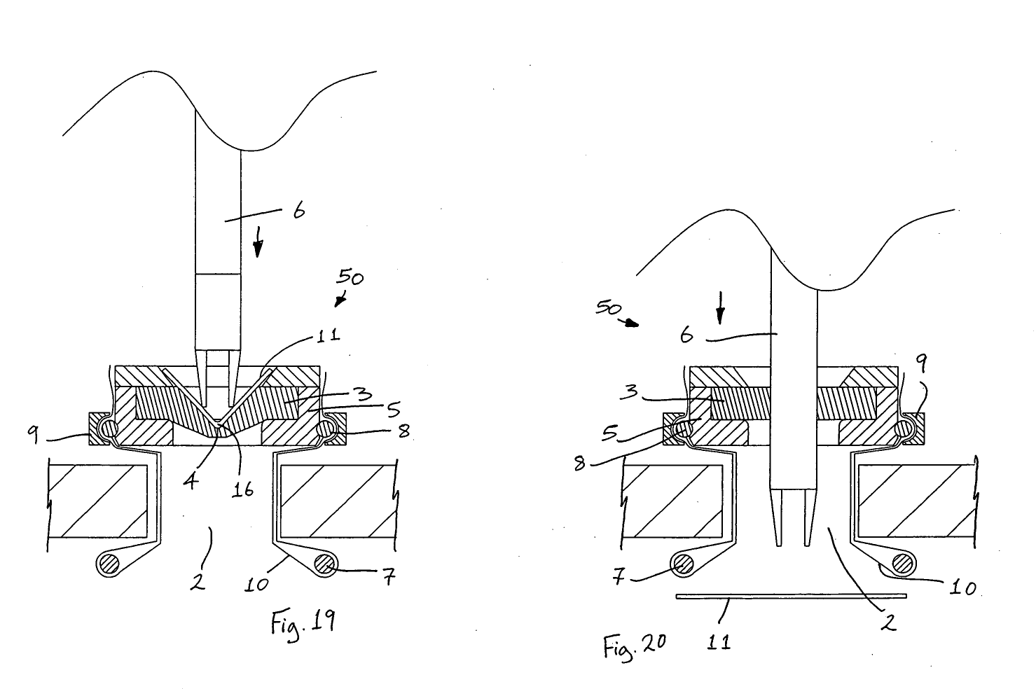

FIG. 17 illustrates the free gel guard 11, for example a disc of plastic film. The instrument 6 hits the gel guard 11 which is forced onto the gel 3 (FIG. 18). The gel guard film 11 finds the pinhole 4 and spreads the gel 3 to allow the instrument 6 to pass through (FIG. 19). The instrument 6 passes easily through with no damage to the gel 3 (FIG. 20).

In FIGS. 21 to 25 there is illustrated another instrument access device 60 according to the invention, which is similar to the device 1 of FIGS. 1 to 9, and similar elements in FIGS. 21 to 25 are assigned the same reference numerals.

In this case the sheet 11 is attached to the seal housing 5 by means of a flexible tether connector 61.

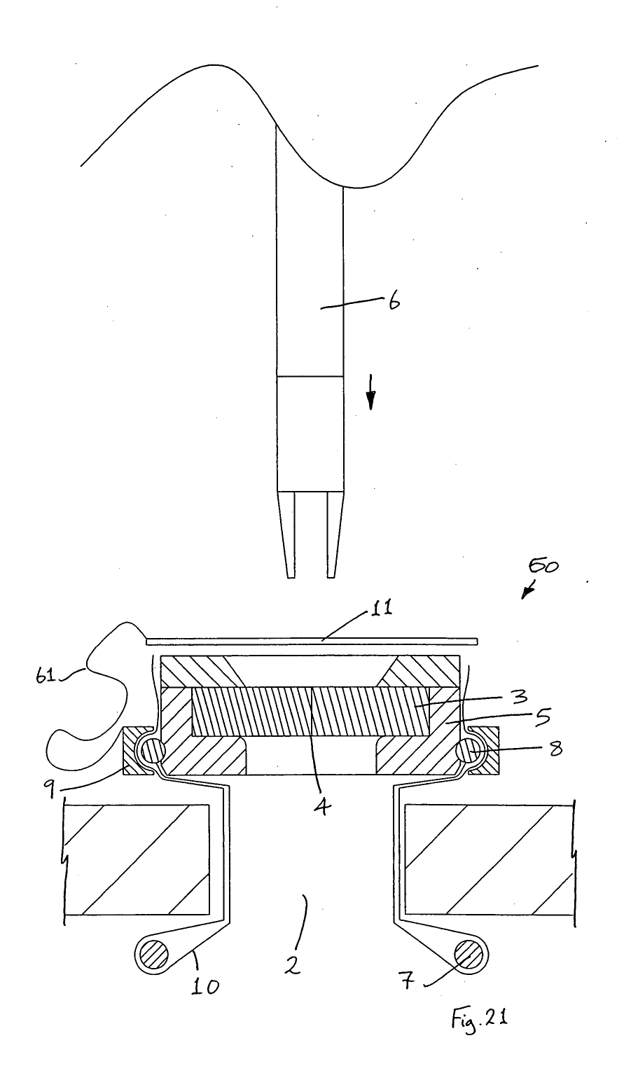

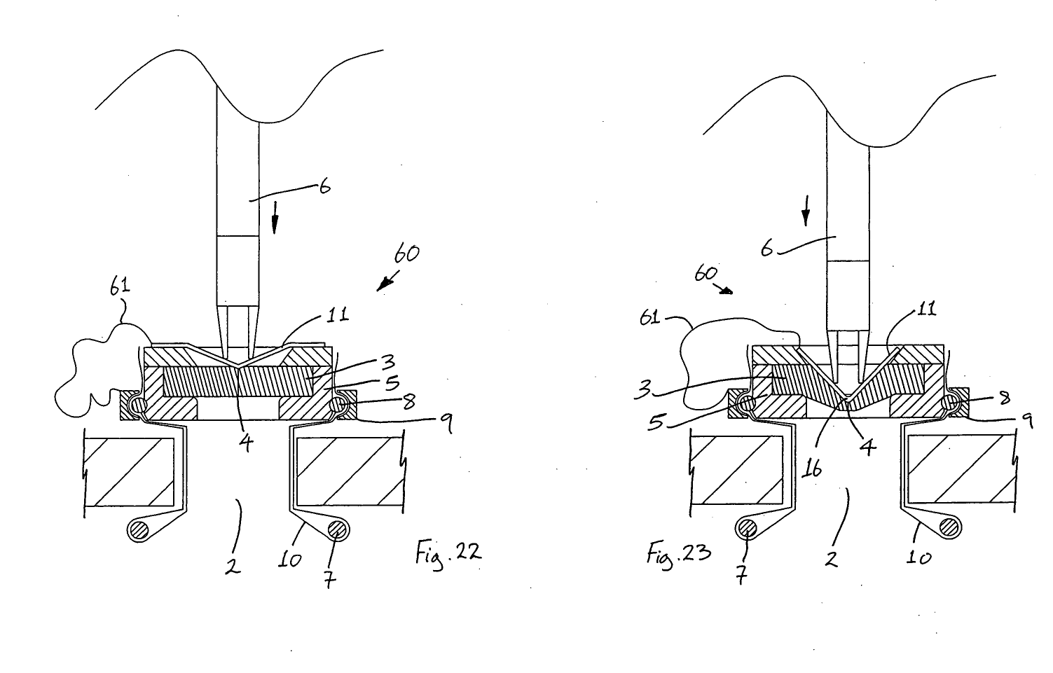

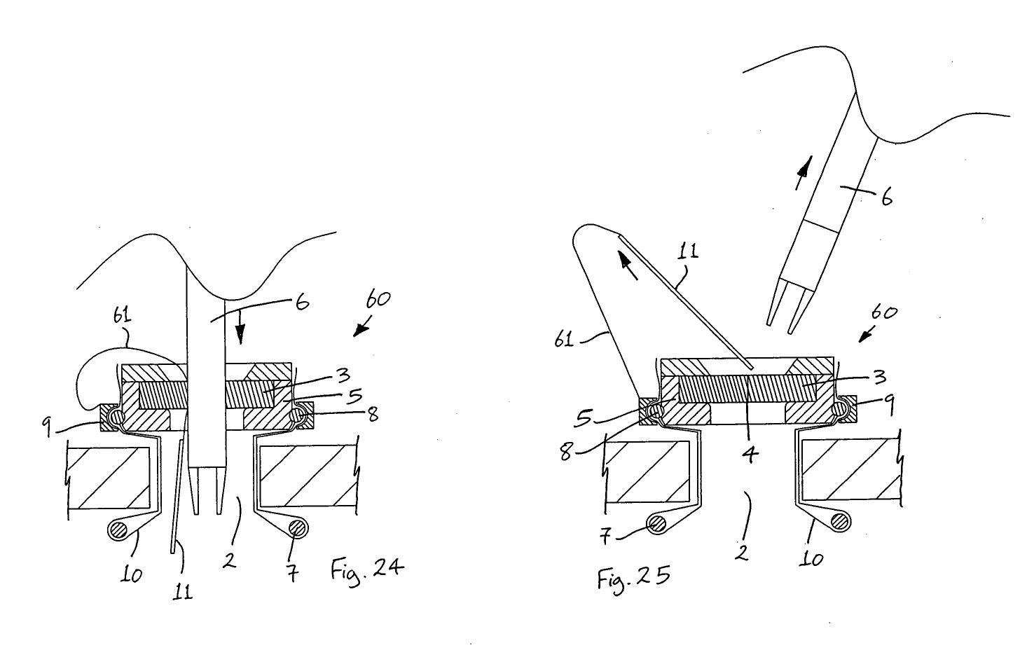

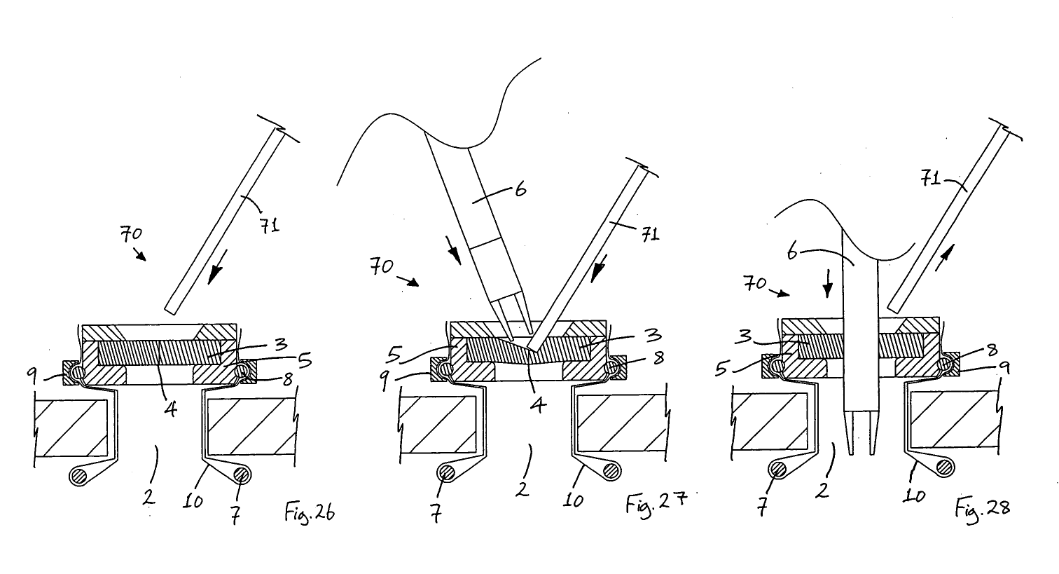

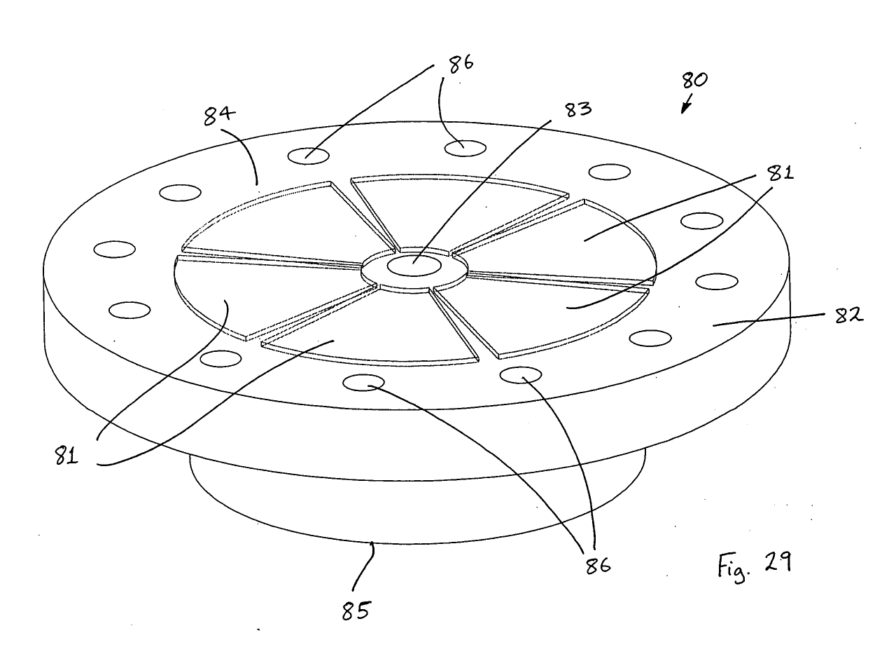

FIG. 21 illustrates the gel guard film 11 and the tether 61. The gel guard film 11 is brought into the abdomen by the instrument 6 (FIG. 24). To retrieve the gel guard film 11, the user pulls on the tether 61, either when the instrument 6 is still in place, or after the instrument 6 has been removed (FIG. 25).

FIGS. 26 to 28 illustrate a further instrument access device 70 according to the invention, which is similar to the device 1 of FIGS. 1 to 9, and similar elements in FIGS. 26 to 28 are assigned the same reference numerals.

In this case the opening element is provided in the form of a rigid rod 71. The rod 71 is detached from the seal housing 5.

The rod 71 operates in the manner of a shoe-horn. The shoe-horn rod 71 finds the pinhole 4 and begins to spread the gel 3 for the instrument 6 (FIG. 27). The shoe-horn rod 71 is removed once the instrument 6 is at least partially through the gel 3 (FIG. 28).

Referring to FIGS. 29 to 31 there is illustrated another instrument access device 80 according to the invention. The device 80 comprises an instrument seal, and six reinforcement elements 81 for reinforcing the seal upon engagement of an instrument with the seal.

The instrument seal comprises a gelatinous elastomeric seal 82 with a lipseal opening 83 extending therethrough. The instrument may be inserted through the opening 83.

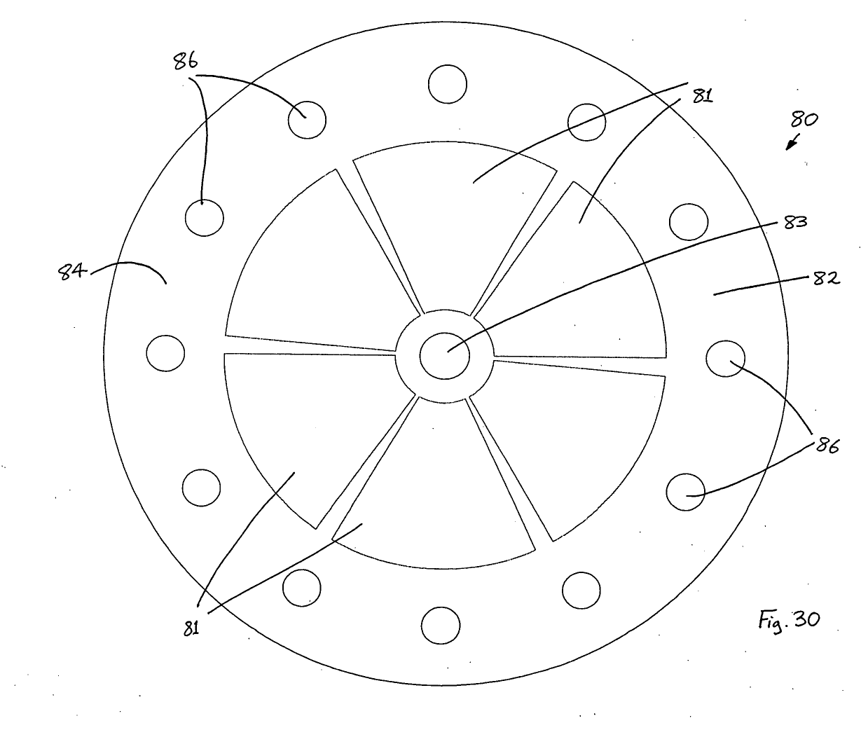

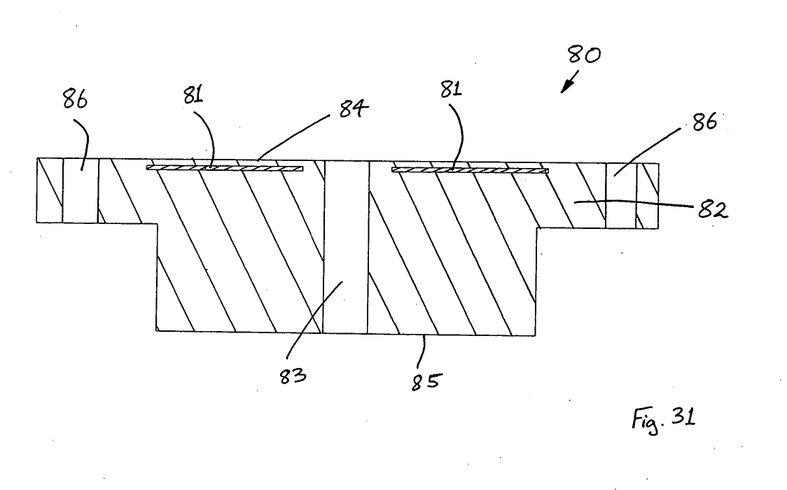

The reinforcement elements 81 are located distally of the proximal end 84 of the seal 82 and proximally of the distal end 85 of the seal 82, embedded within the seal 82 (FIG. 31). The reinforcement elements 81 are located radially outwardly of the opening 83, circumferentially spaced-apart around the opening 83 (FIG. 30). The reinforcement elements 81 are of a more rigid material than the seal 82.

In use, a wound opening is created, and the instrument seal is arranged externally of the wound opening. Upon advancement of the instrument distally, the instrument engages the proximal end 84 of the seal 82. Further advancement of the instrument forces the wall of the opening 83 radially outwardly, as the instrument advances distally though the opening 83 to access the wound interior.

Because of the presence of the reinforcement elements 81 in the seal 82, it is easier for the instrument to pass through the opening 83 without the risk of the instrument inadvertently piercing or ripping the seal 82 to the side of the opening 83.

FIGS. 29 to 31 illustrate the material 81 embedded just below the top surface 84 of the gel lipseal 82. The material 81 protects the gel 82 when an instrument, for example a clip applier, hits the top surface 84 of the lipseal 82. The embedded material 81 helps to open the lipseal hole 83 for the instrument.

FIG. 29 illustrates the gel lipseal valve 82, the embedded material 81, the lipseal hole 83, and pin locating holes 86. FIG. 30 illustrates the embedded material 81, the lipseal hole 83, and the pin locating holes 86. FIG. 31 illustrates the pin locating holes 86, the embedded material 81, and the lipseal hole 83.

The invention is not limited to the embodiments hereinbefore described, with reference to the accompanying drawings, which may be varied in construction and detail.

1. An instrument access device comprising:—a distal anchoring member for insertion into a wound interior;
an elongate member extending proximally from the distal anchoring member to retract laterally the sides of a wound opening;
at least one instrument seal; and
at least one opening element to at least partially open the at least one instrument seal for insertion of an instrument through the at least one instrument seal.

2. A device as claimed in claim 1 wherein at least part of the opening element is movable relative to the seal to open the seal.

3. A device as claimed in claim 2 wherein the opening element is movable distally relative to the seal.

4. A device as claimed in claim 2 wherein the opening element is movable between a rest configuration and an opening configuration.

5. A device as claimed in claim 4 wherein in the opening configuration, at least part of the opening element is substantially pointed.

6. A device as claimed in claim 4 wherein in the opening configuration, at least part of the opening element is substantially tapered.

7. A device as claimed in claim 4 wherein in the opening configuration, at least part of the opening element is substantially conical-shaped.

8. A device as claimed in claim 4 wherein in the rest configuration, the opening element is substantially planar.

9. A device as claimed in claim 1 wherein the opening element is attached to the seal.

10. A device as claimed in claim 9 wherein the device comprises a connector element to attach the opening element to the seal.

11. A device as claimed in claim 1 wherein the opening element is detached from the seal.

12. A device as claimed in claim 1 wherein the opening element comprises a sheet of material.

13. A device as claimed in claim 12 wherein in the opening configuration, a first part of the sheet overlaps a second part of the sheet.

14. A device as claimed in claim 13 wherein the sheet comprises a discontinuity between the first part and the second part.

15. A device as claimed in claim 14 wherein the discontinuity comprises a slit in the sheet.

16. A device as claimed in claim 1 wherein the opening element is substantially flexible.

17. A device as claimed in claim 1 wherein the opening element is substantially rigid.

18. A device as claimed in claim 1 wherein the device comprises a plurality of opening elements.

19. A device as claimed in claim 1 wherein the seal comprises a gelatinous elastomeric material.

20. A device as claimed in claim 1 wherein the seal comprises at least one opening extending therethrough through which an instrument may be inserted.

21. A device as claimed in claim 20 wherein the opening element is configured to at least partially open the opening for insertion of an instrument through the opening.

22. A device as claimed in claim 20 wherein the opening is biased towards a closed configuration.

23. A device as claimed in claim 20 wherein the opening comprises a pinhole opening.

24. An instrument access device comprising:—
at least one instrument seal;
the at least one instrument seal comprising at least one opening extending at least partially therethrough through which an instrument may be inserted; and
at least one reinforcement element to reinforce the at least one instrument seal upon engagement of an instrument with the at least one instrument seal;
the at least one reinforcement element being located radially outwardly of the opening.

25. A device as claimed in claim 24 wherein the reinforcement element is located distally of a proximal end of the seal.

26. A device as claimed in claim 24 wherein the reinforcement element is located proximally of a distal end of the seal.

27. A device as claimed in claim 24 wherein the reinforcement element is at least partially embedded within the seal.

28. A device as claimed in claim 24 wherein the reinforcement element is of a more rigid material than the seal.

29. A device as claimed in claim 24 wherein the seal comprises a gelatinous elastomeric material.

30. A device as claimed in claim 24 wherein the opening comprises a lipseal opening.

31. A device as claimed in claim 24 wherein the device comprises a plurality of reinforcement elements.

32. A device as claimed in claim 31 wherein the reinforcement elements are circumferentially spaced-apart around the opening.

33. A device as claimed in claim 24 wherein the device comprises:—
a distal anchoring member for insertion into a wound interior; and
an elongate member extending proximally from the distal anchoring member to retract laterally the sides of a wound opening.

34. A device as claimed in claim 1 wherein the device comprises a proximal member for location externally of a wound opening.

35. A device as claimed in claim 34 wherein the proximal member comprises a ring member.

36. A device as claimed in claim 34 wherein the proximal member comprises a proximal inner element and a proximal outer element.

37. A device as claimed in claim 36 wherein the elongate member is led between the proximal inner element and the proximal outer element.

38. A device as claimed in claim 36 wherein the proximal inner element and/or the proximal outer element comprises a ring element.

39. A device as claimed in claim 36 wherein the proximal outer element is mounted to the proximal inner element.

40. A method of accessing a wound interior with an instrument, the method comprising the steps of:—
creating a wound opening;
inserting a distal anchoring member into the wound interior;
retracting the sides of the wound opening;
arranging an instrument seal at the wound opening;
at least partially opening the instrument seal; and
inserting an instrument through the instrument seal to access the wound interior.

41. A method as claimed in claim 40 wherein the seal is opened using an opening element.

42. A method as claimed in claim 41 wherein the method comprises the step of moving at least part of the opening element relative to the seal to open the seal.

43. A method as claimed in claim 42 wherein at least part of the opening element is moved distally relative to the seal.

44. A method as claimed in claim 42 wherein the method comprises the step of engaging the instrument with the opening element to move at least part of the opening element relative to the seal.

45. A method as claimed in claim 40 wherein the sides of the wound opening are retracted using an elongate member extending proximally from the distal anchoring member.
